(19) United States
(12) Patent Application Publication
(10) Pub. No.: US 2007/0041921 A1
(43) Pub. Date: Feb. 22, 2007

(54) METHOD FOR RELAXING HAIR

(75) Inventors: Paul H. NEILL, Oak Brook, IL (US); Margie FOWLER, Elgin, IL (US); Monica MEDEIROS (nee BISPO), Campinas SP CEP 13031-000 (BR); Trefor A. EVANS, Lombard, IL (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/464,902

(22) Filed: Aug. 16, 2006

Related U.S. Application Data
(60) Provisional application No. 60/709,400, filed on Aug. 18, 2005.

(51) Int. Cl. A61K 8/19

(52) U.S. Cl. 424/70.2

(57) ABSTRACT

A method of relaxing naturally curly hair that comprises: A) carrying out a hair treatment session that comprises the steps of: i) applying to the hair, an alkaline hair relaxer comprising an active hydroxide, ii) allowing the relaxer to remain on the hair for a total period of less than about 10 minutes; iii) removing the relaxer from the hair; and B) repeating the treatment session described in part (A) to attain a desired degree of hair relaxation, with the interval between individual treatment sessions ranging from about 3 to about 20 days.

This application claims the benefit of U.S. Provisional Application No. 60/1709,400 filed Aug. 18, 2005.

### FIELD OF THE INVENTION

This invention relates to a method for relaxing hair using an alkaline hair relaxer.

### BACKGROUND OF THE INVENTION

Naturally curly or kinky hair is conventionally relaxed or straightened through the use of highly alkaline hair relaxers, i.e., relaxers having a pH of at least about 12, also referred to as “strong base” relaxers. Typically, these alkaline hair relaxers derive their chemical activity from active inorganic and/or organic hydroxides.

Structurally, hair fibers have an exterior sheath known as the cuticle and an interior region or core known as the cortex. Both the core and cuticle are rich in keratin, a complex polypeptide made-up of numerous amino acids, including the thiol-containing amino acid cysteine. Cysteine units in the same or different polypeptide chains can react with one another to form disulfide (cysteine) linkages. The disulfide linkages form intra- and inter-chain cross-linkages, thereby altering the structural configuration of the polypeptide chains. Alkaline hair relaxers function to attack the keratin disulfide bonds.

Without wishing to be bound to theory, two reaction mechanisms have been commonly used to explain the operation of alkaline relaxers. One mechanism involves a bimolecular nucleophilic reaction in which the ionic hydroxide attacks the disulfide linkages directly. The second mechanism involves a beta-elimination reaction that is initiated by the nucleophilic attack of hydroxide ion on the hydrogen atom bonded to the carbon atom that is in a beta-position with respect to the disulfide bond of the cysteine residue. As described in U.S. Pat. No. 6,800,277, this beta elimination reaction results in the formation of a dehydroalanine residue that reacts with either the thiol group of a cysteine residue or the amino group of an alanine residue to form a lanthionine or lysinoalinine residue, respectively.

The longer the treatment with an active-hydroxide containing relaxer, the greater the number of hydroxide ions that are absorbed by the hair cortex, and the greater the straightening effect that is obtained. While treatment times may vary depending upon factors that include the solubility, source and concentration of the active-hydroxide relaxer, in the case of naturally curly hair, in order to remove substantially all curliness and achieve a visibly straight hair configuration, herein referred to as “permanent” relaxation or straightening, it is common practice for the relaxer to remain on the hair for a period of at least about 20 minutes. Shorter treatment periods, result in lesser amounts of disulfide linkages being disrupted and the hair is generally not sufficiently straightened. Unfortunately, owing to its caustic nature, the longer the treatment with a reactive hydroxide-containing relaxer, the greater the potential for damage to the hair cuticle and cortex. This is particularly true of the so-called “lye” type relaxers.

Hair relaxer compositions are described in publications that include the following:

U.S. Pat. No. 4,324,263 discloses a hair straightening and a hair curling process. In the process therein described, the relaxer is a composition preferably formed by reacting in an aqueous medium at least one water-soluble inorganic hydroxide with at least one guanidine salt, the anion of which forms a substantially water-insoluble salt with the cation of the hydroxide. The patent describes the time of treatment with such relaxer formulations as normally being within the range of 5-45 minutes, with the time starting from the first application of the relaxer composition to the hair. The patent further states that generally “this treatment time will be at least 10 minutes, and there is no real upper limit on the time that the composition can remain on the hair, with the above-noted 45 minute time generally being about the greatest length of time that is commercially acceptable to end users. It is greatly preferred to utilize no more than about 30 minutes, preferably [sic] less than 25 minutes, of treatment time, and more preferably the treatment time is in the neighborhood of 20 minutes.”

U.S. Pat. No. 4,390,033 discloses a no-base hair relaxer composition for use under highly alkaline conditions comprising water having dispersed therein about 3 to about 50 weight percent oleaginous material, about 7 to about 25 weight percent emulsifier and about 2 to about 30 weight percent of a lipophilic organically modified hectorite clay gellant. The patent refers to “no-base” formulations as “one package systems in which the aqueous and oleaginous materials are co-emulsified”, as distinguished from “with base” products which are supplied with a separate oleaginous base that is applied as a protective layer prior to the application of relaxer. The patent describes the application of the relaxer to the sectioned hair as taking about 8 minutes, with the total time for smoothing normally taking from about 5 to about 10 minutes, depending up the hair length and thickness. In the exemplified treatment, the relaxer is said to be on the head for about 13 to about 18 minutes prior to being removed by rinsing with warm water.

U.S. Pat. No. 5,376,364 discloses substantially liquid conditioning activators for no-lye hair relaxer systems. In the exemplified relaxer treatment, the application of the relaxer to the sectioned hair is described as taking about 8 minutes, with the total time for smoothing normally taking from about 5 to about 10 minutes, depending up the hair length and thickness. According to this description, the relaxer is on the head for about 13 to about 18 or about 20 minutes prior to being removed by rinsing with warm water.

U.S. Pat. No. 5,476,650 discloses hair relaxer compositions “used in high alkaline conditions comprising at lease [sic] one active hair relaxer ingredient, moisturizers, emollients, and emulsifier, with polymethacrylamidopropyltrimonium chloride included in the formula.” In Example III, the patent describes a treatment wherein the relaxer mixture is left on the hair for a period of between 10 to 28 minutes, depending upon hair texture, timing from the start of application.

U.S. Pat. No. 5,849,277 describes relaxer emulsions prepared using an excess of lithium carbonate and a limited amount of calcium hydroxide as reactive ingredients. The compositions are described to be “highly effective hair relaxer formulations having a very low potential for causing skin irritation during the timeframe (usually no more than 30 minutes) of a traditional relaxer treatment application.”

U.S. Pat. No. 2003/0175233 discloses hair cosmetic agents therein described as permitting “effective relaxation without irritation to the scalp occurring or without the hair becoming damaged.” The hair cosmetic agents described by this application comprise:

(i) polymer obtained by free-radically initiated polymerization of:
(a) 1 to 100% by weight, preferably 2 to 95% by weight of at least one cationic monomer chosen from N-vinylimidazoles and diallylamines, optionally in partially or completely quaternized form,
(b) 0 to 99% by weight of at least one water-soluble monomer different from (a) and
(c) 0 to 50% by weight of at least one further free-radically copolymerizable monomer different from (a) or (b), with subsequent partial or complete quaternization or protonation of the polymer if a nonquaternized or only partially quaternized monomer is used as monomer (a), and

(ii) relaxer.

Customary contact times for these agents are disclosed to be in the range of 10 to 20 minutes.

Relaxer treatments are ordinarily repeated once every 6 to 8 weeks, which period is considered to be a typical relaxation cycle. Subsequent to treatment, the ease with which the relaxed hair may be styled varies, depending upon the phase of the relaxer cycle. For example, within about the first to about the third or fourth week following treatment (corresponding to the period when the hair has retained some “bounce” and before the appearance of a significant amount of new growth) the relaxed or straightened hair is generally perceived by consumers to be relatively easy to style. About three or four weeks after the relaxer treatment, the consumer may begin to use straightening tongs or other styling aids in an attempt to control outgrowth. About five to six weeks into the relaxation cycle, with the lengthening of outgrowth, the hair typically becomes very difficult to style and is hard to control. Approximately two weeks before the commencement of next relaxation cycle, consumers may stop washing their hair to build-up what are perceived to be skin-protecting scalp oils.

It is an object of this invention to provide a method of permanent hair relaxation in which relaxer treatment times are reduced. In at least one embodiment, it is also an object of this invention to provide a method of permanent hair relaxation that, after a desired degree of relaxation is achieved, provides sustained ease of styling between relaxer treatments. It is a further object of this invention to provide, in at least one embodiment, a method of hair relaxation that does not require consumers to change their hair styling habits between relaxation treatments. It is yet another object of this invention to provide users with a kit that enables them to carry out the method of this invention.

### SUMMARY OF THE INVENTION

It has now been found that repeated treatments with a highly alkaline hair relaxer for relatively short application periods, i.e., less than about 10 minutes, can provide straightening that approximates or equals that afforded by a single conventional treatment where the relaxer is typically applied for a period of 20 minutes or more. It has further been found that using shorter application periods and shortening the interval of time between the relaxer treatments can minimize the issues associated with regrowth. It has also been found that increasing the frequency of relaxer treatments in accordance with the method of this invention results in hair having a tensile strength that is generally comparable to or, in some instances, better then that of hair relaxed by means of conventional alkaline treatments which commonly employ longer intervals, typically about 6 to about 8 weeks, between treatment sessions.

In one embodiment this invention relates to a method of relaxing naturally curly hair that comprises:

A) carrying out a hair treatment session that comprises the steps of:
i) applying to the hair, an alkaline hair relaxer comprising an active hydroxide,
ii) allowing the relaxer to remain on the hair for a total period of less than about 10 minutes;
iii) removing the relaxer from the hair; and

B) repeating the treatment session described in part (A) to attain a desired degree of hair relaxation, with the interval between individual treatment sessions ranging from about 3 to about 20 days.

Having attained a desired degree of relaxation, the treatment sessions may be repeated, as needed, to maintain ease of styling and a relaxed hair appearance as the treated hair grows out and new proteins are generated at the fiber base.

In another embodiment, this invention relates to a hair treatment product that comprises, in a single kit;

A) a relaxer composition that provides a source of active hydroxide,

B) optionally, an activator composition,

C) optionally, a neutralizing composition,

D) optionally, a conditioning composition, and

E) instructions teaching the use of the kit components in such a manner that the hair to be straightened is subjected to multiple treatments of reactive hydroxide, for periods lasting less than about 10 minutes, with the interval between treatments ranging from about 3 to about 20 days, until a desired degree of hair relaxation is attained.

### DETAILED DESCRIPTION OF THE INVENTION

Of particular interest are treatments wherein active relaxer is allowed to remain on the hair for a period of less than about 8 minutes, preferably up to about 5 minutes. The optimum interval between individual treatments depends upon factors that include the particular characteristics of the hair, as well as the concentration and source of the reactive hydroxide.

In general, it is desirable for the interval between treatments to be a period of from about 3 to about 20 days, preferably from about 5 to about 15 days, and, in embodiments of particular interest, from about 10 to about 14 days. When the treatment interval extends beyond about 20 days, the presence of regrowth may make it more difficult to achieve a cumulative straightening benefit within an acceptable timeframe. When the treatment interval is less then about 3 days, it may be more difficult for the hair to recover “bounce” or spring.

Once a desired degree of relaxation is attained, the method of the subject invention allows users to maintain same by continuing to repeat the treatment sessions on an as-needed basis; at this point, the interval between individual treatment sessions is preferably from about one to about two weeks. Depending upon factors that include the concentration of active hydroxide, and the period that the relaxer is allowed to remain on the hair, “permanent” relaxation may be achieved in three or fewer relaxer treatment sessions; in some instances, six or more sessions may be needed.

The relaxers suitable for use herein are materials that provide a source of active hydroxide. Exemplary of such materials are “lye” and “no-lye” type relaxers. “Lye” relaxers contain sodium hydroxide; in “no-lye” relaxers the source of active hydroxide is an alkaline agent other than sodium hydroxide, including, for example, other soluble metal hydroxides, for example, alkali metal hydroxides such as lithium and potassium hydroxide, as well as less soluble multivalent metal hydroxides, for example Ca(OH)₂, that are converted, in situ, to soluble active bases, e.g., guanidine hydroxide.

Active hydroxide may be provided by using a less soluble or insoluble multivalent metal hydroxide in conjunction with a complexing agent that dissociates the multivalent metal hydroxide and chelates, sequesters or otherwise ties up the multivalent metal ion and liberates hydroxide ions. Non-limiting examples of less soluble or insoluble metal hydroxides include calcium hydroxide, barium hydroxide, magnesium hydroxide, aluminum hydroxide, cupric hydroxide, strontium hydroxide, molybdenum hydroxide, manganese hydroxide, zinc hydroxide, cobalt hydroxide, and the like. The complexing agents that may be used in the practice of this invention include, but are not limited to, mono-, di-, poly- amino-, and hydroxy-carboxylic acids, mono-, di-, poly-, amino- and hydroxy-sulfonic acids and mono-, di-, poly-, amino- and hydroxy-phosphonic acids. Other suitable complexing agents are chelating agents and sequestering agents. Non-limiting examples of complexing agents include ethylene-diaminetetraacetic acid (EDTA), N-(hydroxyethyl)ethylene diamine triacetic acid, aminotrimethylene phosphonic acid, diethylenetriamine-pentaacetic acid, lauroyl ethylene diamine triacetic acid, nitriloacetic acid, iminodisuccinic acid, tartaric acid, citric acid, N-2-hydroxyethyliminodiacetic acid, and the like. The complexing agents may be used in the form of their corresponding organic and/or inorganic salts, with guanidine salts being exemplary of the corresponding organic salts, and potassium, sodium and/or lithium salts being exemplary of the inorganic salts. U.S. Pat. No. 6,5612,327 is incorporated herein by reference with respect to its disclosure of the activation of multivalent metal hydroxides through the use of a complexing agent.

The alkaline agents that may be used to generate active hydroxide may be chosen from alkali metal hydroxides, alkaline earth metal hydroxides, transition metal hydroxides, Group II hydroxides, Group IV hydroxides, Group V hydroxides, Group VI hydroxides, organic hydroxides, and the like, as well as oxides and salts that are convertible to such hydroxides. Of particular interest in the practice of this invention are relaxers comprising at least one active hydroxide selected from the group consisting of sodium hydroxide, potassium hydroxide, lithium hydroxide and guanidine hydroxide.

The “lye” relaxers used herein typically comprise from about 0.5% to about 2.5% by weight of sodium hydroxide, based on the total weight of the relaxer, with sodium hydroxide concentrations of preference being determined by application period, treatment session intervals, and degree of relaxation desired. In at least one embodiment of this invention, the use of a relaxer comprising sodium hydroxide in an amount of from about 0.7 to about 2.2% percent by weight, more particularly, from about 1.5 to about 2.2% by weight, is of particular interest. In the case of “no-lye” relaxers, the concentration of alkaline agent is generally present in an amount ranging from about 0.5% to about 20% by weight, more particularly from about 0.7 to about 10% by weight, based on the total weight of the relaxer; the concentration of choice being dependent upon the particular alkaline agents present and the solubility thereof. In general, the relaxers used in the compositions of this invention may be formulated to strengths that are comparable to or as low as one quarter the strength of conventional commercially available alkaline relaxers. Using relaxers having lower concentration of active hydroxide may increase the number of relaxer treatments required to attain the desired degree of hair relaxation or strength, however, such treatments may result in hair having improved tensile strength.

As used herein, the term “relaxer” refers to the active hydroxide containing material in the form in which it is applied to the hair, and the term “relaxer composition” refers to an active hydroxide-generating composition that forms a component of the relaxer system kit. Depending upon the active hydroxide component, the relaxer composition may require mixing with a separately packaged activator to generate active hydroxide, as is the case, for example, when guanidine hydroxide is produced by mixing a calcium hydroxide-containing relaxer composition with a guanidine carbonate-containing activator. The term “separately packaged” means packaged in such a manner that the contents thereof do not mix with any other components of the reactor kit until such time as it is intended that they be combined.

To help protect the scalp and skin from the caustic nature of the hydroxide, the relaxer compositions further comprise at least one oleaginous material. Non-limiting examples of suitable oleaginous materials include mineral oils, petrolatum, lanolin, mineral jellies, plant oils, and the like. Such components are well known to those skilled in the art and are commercially available, in cosmetic grades, from numerous suppliers.

In addition to the complexing agents and oleaginous materials referred to above, one or more additives such as are commonly used in hair relaxers may also be present in the relaxers of this invention. Non-limiting examples of these additional additives are, for example, solvents, viscosity modifiers, gellants, surfactants, conditioners, moisturizers, fragrances, masking agents, proteins, preservatives, and mixtures thereof. These additives are well known to those skilled in the art and are commercially available from numerous suppliers. Non-limiting examples of solvents include, for example, water, alkanols, glycerol, glycols and glycol ethers, and aromatic alcohols. In one ore more embodiments of this invention additional additives of interest comprise one or more fatty alcohols and/or glycol ethers thereof, the fatty alcohol preferably being one or more C₁₂-C₂₂ fatty alcohols and/or glycol ethers thereof.

In an embodiment of particular interest, the relaxer used in the method of this invention comprises:

a) at least one reactive hydroxide,

b) at least one oleagenous material,

c) at least one a fatty alcohol and/or a glycol ether thereof the fatty alcohol preferably being a C₁₂-C₂₂ fatty alcohol, and

d) water, preferably deionized water.

The relaxer compositions are typically emulsions or suspensions. In one embodiment, the relaxer compositions are formulated as creams, gels, or foams having Brookfield viscosities of from about 100,000 to about 1,000,000 cps @ 25° C. (TE spindle; 0.5 rpm), with relaxer compositions having Brookfield viscosities of from about 400,000 to about 800,000 cps @ 25° C. (TE spindle; 0.5 rpm) being of particular interest.

In another embodiment, the relaxer compositions of this invention may be formulated into less viscous aqueous hair conditioner bases wherein the presence of oleaginous materials such as mineral oil or petrolatum is reduced or minimized. Aqueous conditioner based relaxer compositions may have having Brookfield viscosities of from about 1,000 to about 100,000 cps @ 25° C. (TE spindle; 0.5 rpm) and, more particularly, from about 2,000 to about 10,000 cps @ 25° C. (TE spindle; 0.5 rpm) being of particular interest. The compositions may be prepared by techniques such as are generally known in the art.

In addition to comprising at least one reactive hydroxide, aqueous conditioner based relaxer compositions comprise conditioning agent and fatty alcohol material. The conditioning agent may be present in such compositions in an amount of from 0.5 to 15%, preferably from 1.5 to 8% by weight, and, in at least one embodiment of interest, from 2 to 6% by weight based on the total weight of the composition. The conditioning agent may comprise a quaternary nitrogen-containing conditioning agent, also referred to as cationic conditioning agents. Cationic conditioning agents suitable for use herein include cationic compounds corresponding to the general formula:
[N(R¹)(R²)(R³)(R⁴)]⁺[X]⁻ (I)
wherein R¹, R², R³, and R⁴ are independently selected from (a) an aliphatic hydrocarbyl group of from 1 to about 22 carbon atoms and (b) hydrogen or an aromatic, alkoxy, polyoxyalkylene, alkylamido, hydroxyalkyl, aryl or alkylaryl group having up to about 22 carbon atoms; and X is a salt-forming anion such as those selected from halide (e.g., chloride, bromide), acetate, citrate, lactate, glycolate, phosphate, nitrate, sulfate, and alkylsulfate radicals, preferably at least one of R¹, R², R³, and R⁴ is an aliphatic hydrocarbyl group having from about 8 to about 22 carbon atoms, preferably alkyl. The aliphatic hydrocarbyl groups can contain, in addition to carbon and hydrogen atoms, ether linkages, and other groups such as amino groups. The longer chain aliphatic hydrocarbyl groups, e.g., those of about 12 carbons, or higher, can be saturated or unsaturated. Moreover, these longer chain groups need not be solely one chain length, rather these groups may be mixtures of different length chains. Salt-forming anions of particular interest are halides, preferably chloride.

Included among such cationic conditioning agents are Formula I compounds as described above wherein R¹, R², R³, and R⁴ are independently selected from (a) an aliphatic hydrocarbyl group as described above and (b) an alkoxy, polyoxyalkylene, alkylamido, hydroxyalkyl, aryl or alkylaryl group as described above. Formula I compounds of particular interest include monoalkyl quats having the general formula described above wherein R₁, R₂ and R₃, which may be the same or different, are C1-C4 alkyl groups and R₄ is a C8 or greater, preferably C14 to C22, hydrocarbyl group; optionally one of the C1-C4 alkyl groups may be replace by an aromatic group, preferably a benzyl group. Other Formula I compounds of interest include dialkyl quats having the general formula described above wherein R₁ and R₂, which may be the same or different, are C1-C4 alkyl group, and R₃ and R₄, which may be the same or different, are C8 or greater, preferably C14 to C22, hydrocarbyl groups, preferably alkyl.

Examples of suitable cationic compounds include: pyridinium chloride, docosyl trimethyl ammonium chloride (behentrimonium chloride), octyltrimethylammonium chloride, dodecyltrimethylammonium chloride, hexadecyltrimethylammonium chloride, octyldimethylbenzylammonium chloride, decyldimethylbenzylammonium chloride, octadecyidimethylbenzylammonium chloride, tallowtrimethylammonium chloride, cocotrimethylammonium chloride, dihexadecyldimethylammonium chloride, dioctadecyldimethylammonium chloride, didodecyidimethylammonium chloride, dimethyldi(hydrogenated tallow) ammonium chloride, PEG-2 oleylammonium chloride.

Other suitable cationic conditioning agents are polymers characterized by the presence of cationic nitrogen groups such as cationic-nitrogen-containing groups, protonated amino groups or mixtures thereof. Such polymers are described in the CTFA Cosmetic Ingredient Dictionary, 3^{rd} edition. The weight average molecular weight of the cationic polymer will preferably range from about 10,000 to about 2,000,000. Quaternary silicone polymers, quaternary polysaccharide polymers, quaternary cellulose derivatives, quaternary starch derivatives and quaternary guar gum derivatives are also exemplary of suitable cationic conditioning agents. The cationic conditioning agent may, but need not, be present as mixtures.

The fatty alcohol material may be present in the aqueous conditioner based relaxer composition in an amount of from 2 to 15 weight percent, preferably from 2 to 12 weight percent and, in one embodiment of particular interest, from 5 to 10 weight percent, based on the total weight of the composition. The “fatty alcohol material” means an alcohol or amine having at least one fatty chain, preferably alkyl, of from about 8 to about 22, preferably from about 12 to about 20, carbon atoms. The fatty material may, but need not be “alkoxylated” (e.g., ethoxylated or propoxylated). The fatty material may but need not be in the form of mixtures. Examples of suitable fatty materials include cetyl alcohol, stearyl alcohol, cetearyl alcohol, stearamidopropyl dimethylamine, ethylene glycol cetyl ether, polyoxyethylene (2) stearyl ether, ceteth 1, steareth 2, ceteareth 20, and the like. The fatty alcohol may, but need not, be present as mixtures.

The aqueous conditioner based relaxer composition may further comprise one or more additional components such as are conventionally present in hair conditioner bases, such as, for example, emulsifiers, suspending agents, additional conditioners, thickening agents, viscosity modifiers, fragrances, and colorants.

In use, the relaxer is applied to and distributed evenly on the hair. To promote the straightening process, the hair may be subjected to a gentle straightening force such as may be achieved by smoothing the hair with the fingers, straightening brush, back of a comb, or the like.

At the conclusion of the treatment period the relaxer is removed, typically by rinsing with water. To neutralize any residual relaxer that may remain on the hair the hair may be further treated with a neutralizing agent such as, for example a neutralizing shampoo. If desired, the relaxed hair may be treated with at least one conditioning agent. Exemplary of such conditioning agents are moisturizers, conditioners and oils.

As used herein, the term “comprising” is meant not to be limiting to any subsequently stated elements but rather to encompass non-specified elements of major or minor functional importance. In other words the listed steps, elements or options need not be exhaustive. Whenever the words “including or “having are used, these terms are meant to be equivalent to comprising as defined above.

Except in the operating and comparative examples, or where otherwise explicitly indicated, all numbers in this description indicating amounts of material, including material ratios, ought to be understood as modified by the word “about”.

The invention is further illustrated by way of the following non-limiting examples. All parts, percentages and proportions referred to here and elsewhere in the specification and the appended claims are by weight, unless specified otherwise.

### EXAMPLES

Relaxer formulations as described in Table 1 were formulated by means of the following procedure:

In a suitably sized first vessel, under moderate agitation, a mixture of all formulation components except the mineral oil, petrolatum and hydroxide, were prepared by staged addition and heated to 82-85° C. under moderate agitation. Meanwhile, in a second vessel, the mineral oil and petrolatum were also heated to 82-85° C. When the contents of both vessels were at 82-85° C., the second vessel was emptied into the first vessel. The resulting mixture was maintained at 82-85° C. under high agitation for 30 minutes. Agitation was then reduced to moderate, and cooling of the reaction mixture was begun. When the vessel contents reached 52° C., hydroxide was added. The mixture was cooled to room temperature over a period of 30 minutes, with continued moderate agitation.

TABLE 1

| Strength | | | |
|---|---|---|---|
| E1 | E2 | E3 | E4 |

| Component | Weight % | | | |
|---|---|---|---|---|
| Petrolatum | 15.00 | 15.00 | 15.00 | 15.00 |
| Mineral Oil | 20.00 | 20.00 | 20.00 | 20.00 |
| Cetyl Stearyl Alcohol | 6.80 | 6.80 | 6.80 | 6.80 |
| Stearyl Alcohol and20 | 1.70 | 1.70 | 1.70 | 1.70 |
| Ceteareth- | | | | |
| Laureth-23 | 0.50 | 0.50 | 0.50 | 0.50 |
| Polysorbate-60 | 1.50 | 1.50 | 1.50 | 1.50 |
| Propylene Glycol | 3.00 | 3.00 | 3.00 | 3.00 |
| NaOH | 2.20 | 1.10 | 0.73 | — |
| Lithium Hydroxide | | | | 0.75 |
| (mono hydrate) | | | | |
| Water (deionized) | Balance | Balance | Balance to | Balance to |
| | to 100% | to 100% | 100% | 100% |

The relaxer formulations were tested on South African hair tresses by means of the following procedure:

1.4 g of relaxer/gram of hair tress were worked into each of five tresses weighing approximately 0.40 gm each,

2. The tresses were smoothed with the back of a comb or the fingers and laid out on foil.

3, The relaxer was allowed to remain on the hair for the applicable treatment period (5 or 20 minutes).

4. Upon completion of the treatment period, the hair tresses were rinsed under warm water until no trace of relaxer was left.

5. The tresses were shampooed with 1 ml standard neutralizing shampoo/tress (approximately 30 seconds/tress), and rinsed with water until all the shampoo was removed,

6. The shampoo treatment was repeated two more times.

7. The tresses were laid on foil and blotted with a towel, and allowed to air dry.

The procedure was repeated for multiple treatment cycles that involved a total number of relaxer applications as indicated in Tables 2 and 3. Untreated tresses were employed as a control. Break stress and % straightening values for the treated and untreated tresses are reported in Tables 2 and 3. The 20 minute treatments reported in Table 3 provide a benchmark against the results obtained with treatments of longer duration.

To evaluate the straightening efficiency of the various relaxer treatments, fibers taken from test tresses of the treated hair (or untreated control) were affixed on a sheet of graph paper and carefully extended until all the kinks and curls were removed. Using the graph paper as a template, the fibers were cut to an extended length of 100 mm. The extension force was then removed and the non-extended length of the fiber was measured. The ratio of the non-extended fiber length to the extended fiber length was multiplied by 100 to yield a value termed the “% straightness”. The “% straightness value” was reported as a mean value for 20 specimens (four from each of the five test tresses), from each of which specimen five extended and non-extended fiber length measurements were taken.

To evaluate the effect of the various relaxer treatments on hair strength, break stress values were obtained from wet tensile testing carried out using an Automated Diastron MTT675 instrument. In these tests, the force to break 50 individual fiber specimens (10 from each of the five test tresses) was measured. The diameter of the individual fibers was also measured to allow the force to be normalized against cross sectional area, that is, break stress=force/cross sectional area. Break stress values of the treated and untreated specimens are reported in Tables 2 and 3 as mean values in units of gram-force/micron².

TABLE 2
5 Minute Relaxer Treatments

| | Untreated | | | | | | | | | |
|---|---|---|---|---|---|---|---|---|---|---|
| | (Control) | | 3X | | 6X | | 15X | | 30X | |
| | Break | % | Break | % | Break | % | Break | % | Break | % |
| | Stress | Straightness | Stress | Straightness | Stress | Straightness | Stress | Straightness | Stress | Straightness |
| E4 | 0.013 | 46 | 0.010 | 69 | 0.011 | 71 | 0.010 | 77 | 0.012 | 79 |
| E3 | 0.014 | 46 | 0.012 | 66 | 0.012 | 69 | 0.010 | 75 | 0.010 | 84 |
| E2 | 0.012 | 46 | 0.012 | 71 | 0.010 | 75 | 0.008 | 82 | 0.063 | 86 |
| E1 | 0.012 | 46 | 0.008 | 91 | 0.008 | 90 | 0.005 | 94 | 0.005 | 96 |

TABLE 3
20 Minute Relaxer Treatments

| | Untreated | | | | | | | |
|---|---|---|---|---|---|---|---|---|
| | (Control) | | 1X | | 2X | | 5X | |
| | Break | % | Break | % | Break | % | Break | % |
| | Stress | Straightness | Stress | Straightness | Stress | Straightness | Stress | Straightness |
| E1 | 0.014 | 46 | 0.007 | 88 | 0.005 | 86 | 0.006 | 92 |

The straightening efficiency and break strength data demonstrates that the 5 minute treatments (3, 6, 15 and 30 relaxer cycles) with E1 provided a straightening effect that was comparable to or slightly better than that of the 20 minute treatments with E1 (1,2 and 5 relaxer cycles). Moreover, even after 30 relaxer cycles, hair that had been given 5 minute E1 treatments had a wet tensile strength that was generally comparable to that of hair that had been given the 20 minute E1 treatment for fewer relaxer cycles (1, 2, and 5 cycles). The straightening efficiency of the 5 minute treatment with E2 and E31 (commencing with the data for 15 and 30 cycles, respectively) began to approximate the performance of one or two 20 minute treatments with the more concentrated E1 composition; the wet tensile properties of the 5 minute treatments with these reduced strength formulations was significantly improved compared to the E1 treatments. In terms of straightening efficiency and wet tensile strength, the five minute quarter strength treatments with lithium hydroxide were generally comparable to that of the five minute, E3 sodium hydroxide treatments.

An aqueous conditioner based relaxer formulation as described in Table 4 was prepared.

TABLE 4
Aqueous Conditioner Based Relaxer Formulation

| Component | Weight % |
|---|---|
| Water (deionized) | to 100 |
| Citric acid (50% active) | 0.3 |
| Stearamidopropyl dimethylamine | 0.75 |
| Dicetyldimonium chloride (68% active) | 3.15 |
| Stearyl alcohol and Ceteareth 20 | 1.5 |
| Cetyl alcohol | 5.4 |
| Disodium EDTA | 0.1 |
| Cyclopentasiloxane | 1.8 |
| Sodium hydroxide | 2 |

What is claimed is:

1. A method of relaxing naturally curly hair that comprises:
A) carrying out a hair treatment session that comprises the steps of:
i) applying to the hair, an alkaline hair relaxer comprising an active hydroxide,
ii) allowing the relaxer to remain on the hair for a total period of less than about 10 minutes;
iii) removing the relaxer from the hair; and
B) repeating the treatment session described in part (A) to attain a desired degree of hair relaxation, with the interval between individual treatment sessions ranging from about 3 to about 20 days.

2. A method as described in claim 1, wherein after a desired degree of relaxation is attained, the treatment session described in part (A) is repeated, as needed, to maintain ease of styling and a relaxed hair appearance.

3. A method as described in claim 1, wherein the treatment session described in part (A) is repeated to maintain the desired degree of hair relaxation, with the interval between individual treatment sessions ranging from about 1 to about 2 weeks, after the desired degree of relaxation has been attained.

4. A method as described in claim 1 wherein following step (A)(iii), the hair is further treated with a neutralizing agent.

5. A method as described in claim 1 wherein following step (A)(iii), the hair is further treated with a conditioning agent.

6. A method as described in claim 1 wherein the relaxer is allowed to remain on the hair for a period of up to about 5 minutes.

7. A method as described in claim 1 wherein the interval between individual treatment sessions ranges from about 5 to about 15 days.

8. A method as described in claim 1 wherein the treatment session is repeated a minimum of 3 times.

9. A method as described in claim 1 wherein the relaxer is a lye-type relaxer.

10. A method as described in claim 1 wherein the relaxer is a no-lye type relaxer.

11. A method as described in claim 1 wherein the relaxer comprises at least one active hydroxide selected from the group consisting of sodium hydroxide, potassium hydroxide, lithium hydroxide and guanidine hydroxide.

12. A method as described in claim 1 wherein the relaxer comprises sodium hydroxide.

13. A method as described in claim 1 wherein the relaxer comprises
a) at least one reactive hydroxide,
b) at least one oleagenous material,
c) at least one C₁₂-C₂₂ fatty alcohol and/or a glycol ether thereof, and
d) water.

14. A hair treatment product that comprises, in a single kit:
A) a relaxer composition that provides a source of active hydroxide,
B) optionally, an activator composition,
C) optionally, a neutralizing composition,
D) optionally, a conditioning composition, and
E) instructions teaching the use of the kit components in such a manner that the hair to be straightened is subjected to multiple treatments of reactive hydroxide, for periods lasting less than about 10 minutes, with the interval between treatments ranging from about 3 to about 20 days, until a desired degree of hair relaxation is attained.

15. A hair treatment product as described in claim 14, wherein the instructions teach that the interval between treatments ranges from about 5 to about 15 days, until a desired degree of hair relaxation is attained.

16. A hair treatment product as described in claim 14, wherein active hydroxide is generated by the reaction of the relaxer composition with an activator.

17. A hair treatment product as described in claim 14 wherein the relaxer composition comprises at least one reactive hydroxide in an aqueous conditioner base.

18. A hair treatment product as described in claim 17 wherein the aqueous conditioner base comprises at least one cationic conditioning agent and at least one fatty material.
